(19) United States
(12) Patent Application Publication
(10) Pub. No.: US 2026/0241207 A1
(43) Pub. Date: Aug. 20, 2026

(54) MOTION TRACKING DEVICE, RADIATION THERAPY SYSTEM, AND MOTION TRACKING METHOD

(71) Applicants: Hitachi, Ltd., Tokyo (JP); NATIONAL UNIVERSITY CORPORATION HOKKAIDO UNIVERSITY, Sapporo-shi, Hokkaido (JP)

(72) Inventors: Ling Fung CHEUNG, Tokyo (JP); Shinichiro FUJITAKA, Tokyo (JP); Takaaki FUJII, Tokyo (JP); Naoki MIYAMOTO, Sapporo-shi, Hokkaido (JP)

(21) Appl. No.: 19/159,697

(22) PCT Filed: Feb. 5, 2024
(86) PCT No.: PCT/JP2024/003699

§ 371 Date: Aug. 26, 2025

(30) Foreign Application Priority Data
Apr. 11, 2023 (JP) 2023-064449

(51) Int. Cl. A61N 5/10

(52) U.S. Cl. CPC A61N 5/107; A61N 2005/1062

(57) ABSTRACT

A motion tracking device for tracking a motion of a target and a tissue in a particular region includes: a motion estimator configured to acquire an estimated 3D motion estimating a three-dimensional motion of the target and the tissue in real time, and an estimated 2D motion estimating a two-dimensional motion of the target and the tissue in real time; an image acquisition unit configured to acquire a reference 2D image that is a two-dimensional image of the particular region at a predetermined reference point of time, and a real-time 2D image that is a two-dimensional image of the particular region in real time; an image simulator configured to generate a pseudo real-time 2D image that simulates a two-dimensional image of the particular region in real time using the estimated 2D motion and the reference 2D image; and an estimation corrector configured to correct the estimated 3D motion.

### TECHNICAL FIELD

The present disclosure relates to a radiation therapy technique.

### BACKGROUND ART

In a radiation therapy, ionizing radiation is applied to a target. For example, a high energy particle beam is directed to a tumor region to kill cancer cells. In order to maximize effects of therapy and to minimize side effects of the therapy, it is important to focus radiation on a tumor while preserving healthy tissues surrounding the tumor. However, a human body moves due to respiration and hence, there may be a case where the accuracy of a therapeutic beam is lowered. Accordingly, the motion management is required to deliver an accurate dose of radiation to a tumor with certainty.

A real-time medical imaging technique, such as an X-ray imaging technique, provides information on a real-time motion of body tissues. Such information can be used to guide a therapy beam and to accurately deliver a dose to a tumor. Usually, in real-time medical imaging, a contrast of a tumor is low and hence, there may be a case where a fiducial marker is inserted into a body of a patient by surgery. Although the use of a fiducial marker in the body improves the accuracy of tracking a motion of a tumor, the fiducial marker itself is invasive and, further, there also exists a concern that the fiducial marker moves in a body of a patient.

PTL 1 discloses a method for tracking real-time motion of a target without using a fiducial marker. This method generates an image indicating a target region in advance, and makes a generated image match with a real-time image. For example, a digital reconstructed image (DRR) is first generated from a CT image before a therapy. Next, an optimal matching is identified between a real-time X-ray image taken during therapy and the DRR. The position of the tumor is determined via a corresponding CT image.

PTLs 2 and 3 disclose a markerless tracking method using a technique of estimating a three-dimensional (3D) motion of a tumor from a 2D motion obtained from an image of a 2D magnetic resonance imaging (MRI) or the like. In PTL 2, a transform model is constructed by linear regression, and a relationship between a two-dimensional motion and a three-dimensional motion is specified. In PTL 3, the relationship between a two-dimensional motion and a three-dimensional motion is specified by machine learning.

### CITATION LIST

### Patent Literature

PTL 1: JP 2006-515187 A

PTL 2: JP 2017-537717 A

PTL 3: JP 2022-513427 A

### SUMMARY OF INVENTION

### Technical Problem

A method matching based on matching such as the method disclosed in PTL 1 can be applied to the therapy of a tumor at a specified position, such as a lung, where a contrast between the tumor obtained by a digitally reconstructed radiograph (DRR) and the tumor obtained by a real-time X-ray image is sufficient to ensure effective matching of these images. However, with respect to some tumors such as a tumor in a pancreas, it is difficult to obtain a sufficient contrast in a region around the tumor region between a DRR and a real-time X-ray image and hence, an effective matching of the images cannot be obtained. Further, a DRR is usually generated by projecting a CT image to a two-dimensional (2D) plane and hence, features in a DRR and features in a real-time x-ray images do not necessarily match. Due to such reasons, under current circumstances, the utilization of a markerless tracking method based on matching is limited.

Both the methods described in PTL 2 and PTL 3 establish the relationship between a 2D motion and a 3D motion based on prior knowledge in a learning stage. However, there is a possibility that the relationship between the 2D motion and the 3D motion changes also during therapy. Accordingly, in these methods based on the prior knowledge, there is a possibility that the accurate conversion from the 2D motion to the 3D motion is not performed.

It is an object of the present disclosure to provide a technique capable of tracking the motion of a tumor with high accuracy based on a real-time image without using a marker.

### Solution to Problem

A motion tracking device according to one aspect of the present disclosure is a motion tracking device for tracking a motion of a target and a tissue in a particular region. The motion tracking device includes: a motion estimator configured to acquire an estimated 3D motion estimating a three-dimensional motion of the target and the tissue in real time, and an estimated 2D motion estimating a two-dimensional motion of the target and the tissue in real time; an image acquisition unit configured to acquire a reference 2D image that is a two-dimensional image of the particular region at a predetermined reference point of time, and a real-time 2D image that is a two-dimensional image of the particular region in real time; an image simulator configured to generate a pseudo real-time 2D image that simulates a two-dimensional image of the particular region in real time using the estimated 2D motion and the reference 2D image; and an estimation corrector configured to correct the estimated 3D motion based on a comparison between the pseudo real-time 2D image and the real-time 2D image.

### Advantageous Effects of Invention

According to one aspect of the present disclosure, even if the contrast of a real-time image is not clear based on the real-time image, the motion of a tumor can be tracked with high accuracy without a marker.

### BRIEF DESCRIPTION OF DRAWINGS

FIG. 1 is a configurational diagram illustrating an example of a particle beam therapy system according to the present embodiment.

FIG. 2 is an arrangement view illustrating a schematic arrangement of a patient and a therapy table as viewed from a gantry rotation axis.

FIG. 3 is a block diagram provided for explaining a process of estimation of a motion performed by a motion tracking device.

FIG. 4 is a view schematically illustrating a 3D DVF and a 2D DVF.

FIG. 5 is a view provided for describing an example of processing of acquiring a 2D DVF from a 3D DVF.

FIG. 6 is a view provided for describing another example of processing of acquiring a 2D DVF from a 3D DVF.

FIG. 7 is a flowchart illustrating a series of processing of the particle beam therapy system.

### DESCRIPTION OF EMBODIMENTS

Hereinafter, an embodiment of the present invention will be described in detail with reference to the drawings.

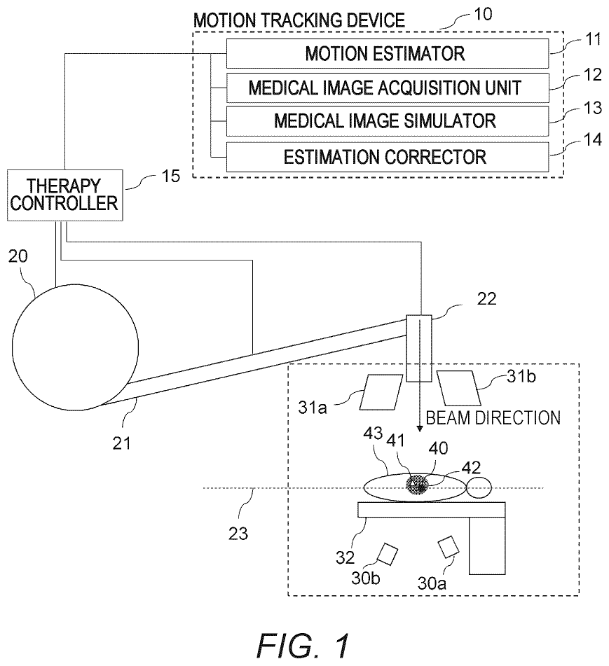

FIG. 1 is a configurational diagram illustrating an example of a particle beam therapy system according to the present embodiment. The particle beam therapy system of the present embodiment is a therapy system that performs proton beam therapy (PBT) as an example.

To describe with reference to FIG. 1, the particle beam therapy system includes a motion tracking device 10, a therapy controller 15, an accelerator 20, a beam transport system 21, a gantry 22, a pair of X-ray sources 30, and a pair of X-ray imaging detectors 31. The tracking device 10 includes a motion estimator 11, a medical image acquisition unit 12, a medical image simulator 13, and an estimation corrector 14.

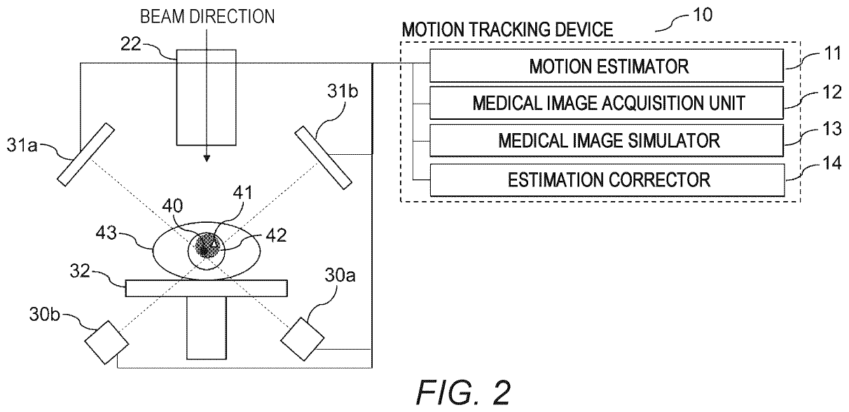

FIG. 2 is an arrangement view illustrating a schematic arrangement of a patient and a therapy table as viewed from a gantry rotation axis. In FIG. 2, a patient 43 and a therapy table 32 as viewed in the direction of the gantry rotation axis 23 shown in FIG. 1 are illustrated.

The patient 43 is placed on the therapy table 32. The gantry 22 rotates about an axis of a gantry rotation axis 23, and can change the beam direction of the particle beam irradiated to the patient 43. The X-ray sources 30a and 30b emit X-rays that pass through the patient 43 to image portions of the body of the patient 43 that include specific regions using the X-ray imaging detectors 31a and 31b, respectively. The specific region is specifically a region of interest (hereinafter also referred to as “ROI”) 42. The ROI 42 includes a target 40 and tissues. The target 40 is a cancer cell group that is a target to which a particle beam is irradiated. The tissue is a group of cells that perform specific functions in the human body.

In one example of the operation of the particle beam therapy system, a charged particle beam having an appropriate energy is extracted from the accelerator 20, and is transported to the gantry 22 via the beam transport system 21. The gantry 22 rotates about the patient 43, and can direct a therapeutic beam to the target 40 at various angles. The accelerator 20, the beam transport system 21, and the gantry 22 are controlled by the therapy controller 15 based on a therapy plan and a motion of the patient 43 estimated by the motion tracking device 10.

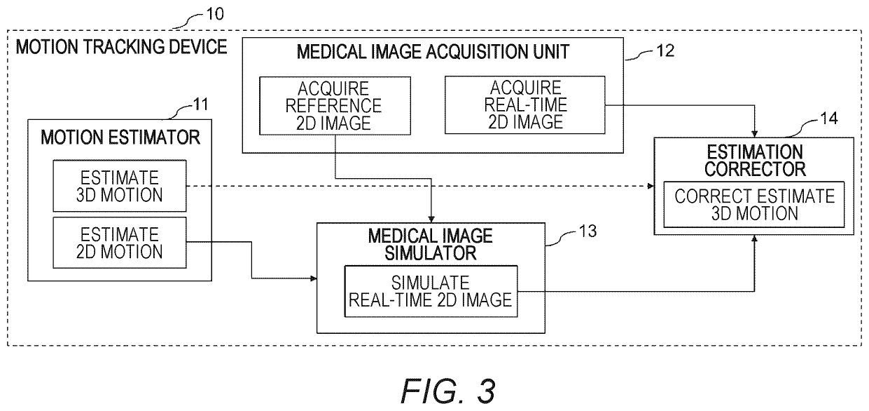

FIG. 3 is a block diagram provided for explaining a process of estimation of a motion performed by the motion tracking device.

Prior to real-time measurement during the therapy applied to the patient 43, the medical image acquisition unit 12 acquires a two-dimensional image at a reference point of time (hereinafter the image being also referred to as a “reference 2D image”). The reference 2D image is a two-dimensional image where breathing is acquired at a point of time of a predetermined phase. Hereinafter, the predetermined phase is also referred to as a reference phase, and the point of time of the reference phase is also referred to as a reference point of time. For example, a point of time at which a patient has completely exhaled is set as the reference phase.

The motion estimator 11 estimates a real-time three-dimensional motion and a two-dimensional motion of tissues in the ROI 42 using a motion model constructed in advance. Hereinafter, the estimated three-dimensional motion is also referred to as an “estimated 3D motion”, and the estimated two-dimensional motion is also referred to as an“ estimated 2D motion”.

The medical image simulator 13 generates a pseudo real-time two-dimensional image (hereinafter also referred to as “pseudo real-time 2D image”) using the reference 2D image and the estimated 2D motion. For example, a pseudo real-time 2D image may be generated by deforming the reference 2D image in accordance with the estimated 2D motion. The medical image simulator 13 generates a pseudo real-time 2D image on a plurality of planes respectively.

Thereafter, when the real-time measurement is performed in actual therapy, the medical image acquisition unit 12 acquires a real-time actual two-dimensional image (hereinafter also referred to as “real-time 2D image”). The medical image acquisition unit 12 acquires real-time 2D images on a plurality of planes.

The estimation corrector 14 compares a plurality of pseudo real-time 2D images generated by the medical image simulator 13 with a plurality of real-time 2D images acquired by the medical image acquisition unit 12, and corrects an estimated 3D motion estimated in advance by the motion estimator 11 in a three-dimensional space based on the comparison result. By correcting the estimated 3D motion on the three-dimensional space based on a differential between the pseudo real-time 2D image and the real-time 2D image, the estimated 3D motion will more accurately represent the current actual target 40 and the motion of the tissues.

The motion model used in the motion estimator 11 described above can be, as an example, constructed based on a 4DCT image that is imaged in advance during a therapy plan and/or a 4D cone beam CT (4-dimensional cone-beam computed tomography: 4DCBCT) image that is imaged before or during the therapy. For example, a motion model may be constructed by performing a principal component analysis (PCA) on a 4DCT image and/or a 4DCBCT image. As another example, the motion model may be constructed by performing machine learning using a 4DCT image and/or a 4DCBCT image as learning data. The 4DCT image is a four-dimensional CT image representing a stereoscopic change with time. The 4DCBCT image is a cone beam CT image representing a stereoscopic change with time. The motion model takes the measured motion of a surrogate 41 as an input, and outputs an estimated 3D motion that estimates the three-dimensional motion of tissues in the ROI 42. The estimated 2D motion can be calculated, for example, from the estimated 3D motion. The estimated 3D motion and the estimated 2D motion can be represented, as an example, in the form of a deformation vector field (DVF). Hereinafter, the three-dimensional DVF is also referred to as a 3D DVF, and the two-dimensional DVF is also referred to as a 2D DVF. These motions represent three-dimensional and two-dimensional motions, respectively.

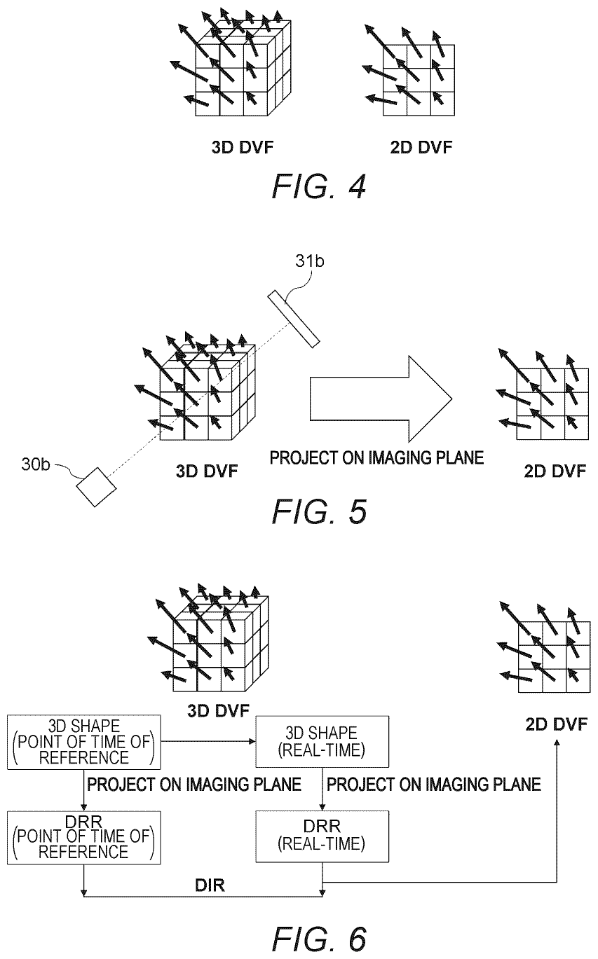

FIG. 4 is a view schematically illustrating the 3D DVF and the 2D DVF.

With reference to FIG. 4, in the 3D DVF, arrows representing displacement vectors in three-dimensional displacements are added to each voxel. These arrows indicate a three-dimensional displacement at the specific time of the specific positions in the ROI with respect to the positions at the reference point of time. In the 2D DVF, arrows representing displacement vectors of two-dimensional displacement are added to each pixel. It can be considered that FIG. 4 illustrates a weighted average motion of the tissues projected onto a plane along a projection axis at a specific time at the specific position with respect to the position at the reference point of time. The directions of the vectors of the DVF can also be reversed such that the directions are directed toward voxels or pixels.

FIG. 5 is a view provided for describing an example of processing of acquiring a 2D DVF from a 3D DVF. In the present example, the 3D DVF is projected onto a two-dimensional plane on which the X-ray imaging detector 31b detects an image. A weighted average is applied to the three-dimensional vectors of the voxels present along the projection axis so that three-dimensional vectors are converted into two-dimensional vectors of the corresponding pixels on the plane of the image detector.

FIG. 6 is a view provided for describing another example of processing of acquiring a 2D DVF from a 3D DVF. In this example, first, a 3D DVF is used so as to render a 3D volume that shows a three-dimensional shape of tissues or the like. Next, a digitally reconstructed image (hereinafter also referred to as “DRR”) is generated by projecting the 3D volume onto a two-dimensional plane of the X-ray imaging detector 31b. A 2D DVF is then generated by deformably aligning the DRR to the DRR at the reference point of time by performing the non-rigid image registration (DIR).

In the present embodiment, the example where the estimated 2D motion is acquired from the estimated 3D motion has been described. However, the present disclosure is not limited to such an example. As another example, as the motion models, a three-dimensional motion model for estimating the three-dimensional motion of the tissues and a two-dimensional motion model for estimating a two-dimensional motion of the tissues may be constructed, and these models may be used. The two-dimensional motion model can be constructed using two-dimensional medical images. A 2D DVF can be generated by the two-dimensional motion model, and a real-time 2D motion can be described.

The real-time 2D motion of the estimated real-time 2D DVF is used to deform the reference 2D image imaged at the reference point of time. The reference 2D image corresponds to the 3D volume at the reference point of time. The reference 2D image deformed using the real-time 2D motion is provided as a pseudo real-time 2D image that simulates the real-time 2D image imaged during therapy.

By comparing the pseudo real-time 2D image with the actually measured real-time 2D image, correction information for correcting the estimated 3D motion that is generated in the motion estimator 11 is generated.

As an example, two-dimensional correction vectors are obtained as a result of strictly aligning the pseudo real-time 2D image with the actually measured real-time 2D image. Next, three-dimensional correction vectors for correcting the estimated 3D motion that is generated in advance are generated by reversely projecting the two-dimensional correction vectors in a three dimensional manner.

As another example, at a result of deforming a pseudo real time 2D image such that the pseudo real time 2D image is aligned with the actually measured real time2D image, a 2D DVF is acquired. The 2D DVF can be converted to a 3D DVF by a predetermined conversion process. The 3D DVF is applied to the correction of the pre-generated estimated 3D motion.

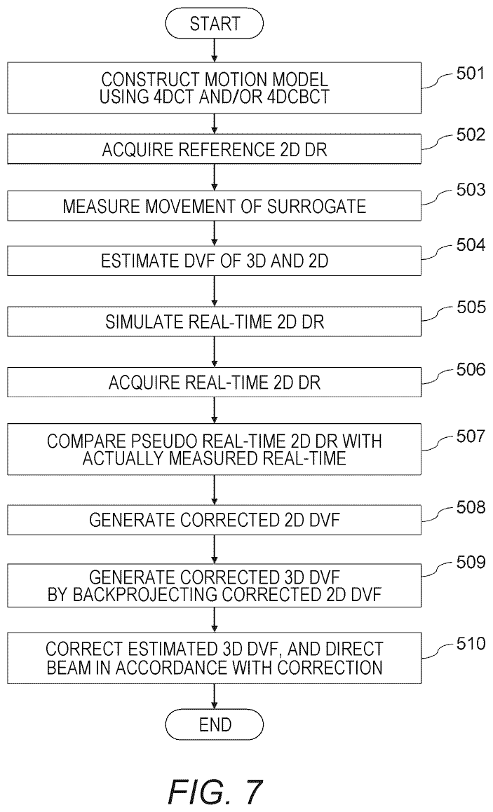

FIG. 7 is a flowchart illustrating a series of processing of the particle beam therapy system.

In step 501, a motion model is constructed using information from the 4DCT and information from the 4DCBCT prior to therapy.

In step 502, a reference digital radiography (DR) is imaged at the same timing as imaging a 4DCBCT image in a reference phase such that the correspondence between the motion model and the reference DR is established.

In step 503, during therapy, the motion of the diaphragm, which is used as a surrogate, is measured, and is used as an input for the motion model. Next, the motion model generates a real-time 3D DVF (hereinafter also referred to as “estimated real-time 3D DVF”) that estimates the motion of the tissues in the ROI.

In step 504, a real-time 3D volume is generated using the estimated real-time 3D DVF. Further, the real-time 3D volume is projected onto a two-dimensional plane to generate a real-time DRR. Further, with respect to the real-time DRR, non-rigid image registration (DIR) is performed on the reference DRR that is generated from the reference 3D volume thus generating estimated real-time 2D DVF. In this embodiment, non-rigid image registration is used as an example. However, the present invention is not limited to such an example, and rigid image registration may be used.

In step 505, the estimated real-time 2D DVF is applied to the reference DR to generate a pseudo real-time DR (hereinafter also referred to as “pseudo real-time DR”).

Thereafter, in step 506, the measurement of the real-time DR is actually performed.

Then, in step 507, the pseudo real-time DR is compared with the actually measured real-time DR.

Next, in step 508, based on a result of the comparison in step 507, a corrected 2D DVF that describes the displacement of each pixel between two images is generated.

In step 509, the corrected 2D DVF is back-projected in three dimensions so as to generate a corrected 3D DVF that represents a real-time correction of the estimated 3D DVF generated in advance.

In step 510, the estimated real-time 3D DVF is corrected using the corrected 3D DVF, and the position of a tumor and tissues is determined using the corrected estimated real-time 3D DVF. The determined information on the position of the tumor and tissues is sent to the therapy controller 15, and the therapy controller 15 performs the orientation of the therapy beam corresponding to the information on the position.

As another example, more accurate 3D DVF may be generated by repeating the above-mentioned correction plural times, and the position of the tumor and the tissues may be determined using the estimated real-time 3D DVF that is corrected more accurately. After the estimated real-time 3D DVF is corrected in step 510, the processing returns to step 504. Then, the iterative processing that the real-time 3D volume and the real-time 2D volume are generated using the corrected estimated real-time 3D DVF is performed. With such processing, the corrected 2D DVF generated in step 508 and the corrected 3D DVF generated in step 509 can be brought close to zero. With such processing, although the calculation time necessary for performing the processing is increased, the more accurate position of the tumor and tissues can be obtained by more accurate 3D DVF.

The above-described embodiments of the present invention are provided for exemplifying the present invention, and the embodiments are not intended to limit the scope of the present invention only to those embodiments. A person skilled in the art can implement the present invention in various other modes without departing from the scope of the present invention. The matters described below are also included in the technical scope of the present disclosure. However, the matters included in the present disclosure are not limited to the matters described below.

### (Item 1)

A motion tracking device for tracking a motion of a target and a tissue in a particular region includes: a motion estimator configured to acquire an estimated 3D motion estimating a three-dimensional motion of the target and the tissue in real time, and an estimated 2D motion estimating a two-dimensional motion of the target and the tissue in real time; an image acquisition unit configured to acquire a reference 2D image that is a two-dimensional image of the particular region at a predetermined reference point of time, and a real-time 2D image that is a two-dimensional image of the particular region in real time; an image simulator configured to generate a pseudo real-time 2D image that simulates a two-dimensional image of the particular region in real time using the estimated 2D motion and the reference 2D image; and an estimation corrector configured to correct the estimated 3D motion based on a comparison between the pseudo real-time 2D image and the real-time 2D image.

According to the above-mentioned configuration, the estimated 3D motion is corrected based on a comparison between the pseudo real-time 2D image generated by deforming the reference 2D image in accordance with the estimated 2D motion and the current real-time 2D image. Accordingly, even if the contrast of the real-time 2D image is not clear, the motion of the target can be tracked with high accuracy without a marker.

### (Item 2)

In the motion tracking device described in the item 1, the motion estimator specifies the estimated 3D motion and the estimated 2D motion by using a motion model constructed based on an image imaged in advance.

With such a configuration, the motion estimated based on the prior knowledge is corrected based on the comparison between the real-time image and the pseudo image based on the estimated motion. Accordingly, even if the motion during the therapy changes from the prior estimation, the motion of the tumor can be tracked with high accuracy.

### (Item 3)

In the motion tracking device described in the item 1, the motion estimator estimates the estimated 3D motion as a three-dimensional vector field, and estimates the estimated 2D motion as a two-dimensional vector field.

With such a configuration, by estimating the motion as a vector field, the motion can be estimated as information that is easily processed by an arithmetic operation.

### (Item 4)

In the motion tracking device described in the item 3, the motion estimator generates a two-dimensional vector field of the estimated 2D motion by projecting the three-dimensional vector field of the estimated 3D motion onto a two-dimensional plane.

With such a configuration, by generating the two-dimensional vector field of the estimated 2D motion by projecting the three-dimensional vector field of the estimated 3D motion onto the two-dimensional plane, the information on the estimated 2D motion can be acquired with a small amount of arithmetic operation.

### (Item 5)

In the motion tracking device described in the item 3, the motion estimator generates a two-dimensional vector field indicating a motion in a two-dimensional shape in a deformed image obtained by deforming a projection image obtained by projecting the three-dimensional shape at the reference point of time onto a two-dimensional plane by non-rigid image registration such that the projection image is made to match a projection image obtained by projecting a three-dimensional shape based on the estimated 3D motion onto the two-dimensional plane, as a two-dimensional vector field of the estimated 2D motion.

With such a configuration, information on the two-dimensional vector field of the estimated 2D motion can be obtained with high accuracy.

### (Item 6)

In the motion tracking device described in the Item 1, the estimation corrector calculates a two-dimensional correction vectors for aligning the pseudo real-time 2D image with the real-time 2D image with respect to a plurality of two-dimensional planes, generates a three-dimensional correction vectors based on the plurality of calculated two-dimensional correction vectors, and corrects the estimated 3D motion using the three-dimensional correction vectors.

With such a configuration, the three-dimensional correction vectors are generated based on the two dimensional correction vectors, and the estimated 3D motion is corrected using the three-dimensional correction vectors. Accordingly, the actual motion of the target and the tissue can be expressed with high accuracy.

### (Item 7)

In the motion tracking device described in the item 1, the motion estimator iterates the correction of the estimated 3D motion by calculating the estimated 3D motion and the estimated 2D motion of the target and the tissue using the estimated 3D motion corrected by the estimation corrector.

With such a configuration, the pseudo real-time 2D image reproduces the real-time 2D image more accurately. Accordingly, the estimated 3D motion can be performed more accurately.

### REFERENCE SIGNS LIST

10 motion tracking device
11 motion estimator
12 medical image acquisition unit
12 medical image acquisition unit
13 medical image simulator
14 estimation corrector
15 therapy controller
20 accelerator
21 beam transport system
22 gantry
23 gantry rotation axis
30 X-ray source
30a X-ray source
30b X-ray source
31 X-ray image detector
31a X-ray image detector
31b X-ray image detector
32 therapy table
40 target
41 surrogate
42 region of interest
43 patient

1. A motion tracking device for tracking a motion of a target and a tissue in a particular region, the motion tracking device comprising:
a motion estimator configured to acquire an estimated 3D motion estimating a three-dimensional motion of the target and the tissue in real time, and an estimated 2D motion estimating a two-dimensional motion of the target and the tissue in real time;
an image acquisition unit configured to acquire a reference 2D image that is a two-dimensional image of the particular region at a predetermined reference point of time, and a real-time 2D image that is a two-dimensional image of the particular region in real time;
an image simulator configured to generate a pseudo real-time 2D image that simulates a two-dimensional image of the particular region in real time using the estimated 2D motion and the reference 2D image; and
an estimation corrector configured to correct the estimated 3D motion based on a comparison between the pseudo real-time 2D image and the real-time 2D image.

2. The motion tracking device according to claim 1, wherein the motion estimator specifies the estimated 3D motion and the estimated 2D motion by using a motion model constructed based on an image imaged in advance.

3. The motion tracking device according to claim 1, wherein the motion estimator estimates the estimated 3D motion as a three-dimensional vector field, and estimates the estimated 2D motion as a two-dimensional vector field.

4. The motion tracking device according to claim 3, wherein the motion estimator generates a two-dimensional vector field of the estimated 2D motion by projecting the three-dimensional vector field of the estimated 3D motion onto a two-dimensional plane.

5. The motion tracking device according to claim 3, wherein the motion estimator generates a two-dimensional vector field indicating a motion in a two-dimensional shape in a deformed image obtained by deforming a projection image obtained by projecting the three-dimensional shape at the reference point of time onto a two-dimensional plane by image registration such that the projection image is made to match a projection image obtained by projecting a three-dimensional shape based on the estimated 3D motion onto the two-dimensional plane, as a two-dimensional vector field of the estimated 2D motion.

6. The motion tracking device according to claim 1, wherein the estimation corrector calculates a two-dimensional correction vector for aligning the pseudo real-time 2D image with the real-time 2D image with respect to a plurality of two-dimensional planes,
generates a three-dimensional correction vector based on the plurality of calculated two-dimensional correction vectors, and
corrects the estimated 3D motion using the three-dimensional correction vector.

7. The motion tracking device according to claim 1, wherein the motion estimator iterates the correction of the estimated 3D motion by calculating the estimated 3D motion and the estimated 2D motion of the target and the tissue using the estimated 3D motion corrected by the estimation corrector.

8. A radiation therapy system comprising:
the motion tracking device according to claim 1; and
a therapy controller that delivers therapeutic radiation to the target based on the 3D motion estimated/corrected by the tracking device.

9. A motion tracking method for tracking a motion of a target and a tissue in a particular region, the method configured to allow a computer to perform steps of:
acquiring an estimated 3D motion estimating a three-dimensional motion of the target and the tissue in real time, and an estimated 2D motion estimating a two-dimensional motion of the target and the tissue in real time;
acquiring a reference 2D image that is a two-dimensional image of the particular region at a predetermined reference point of time, and a real-time 2D image that is a two-dimensional image of the particular region in real time;
generating a pseudo real-time 2D image by deforming the reference 2D image in accordance with the estimated 2D motion; and
correcting the estimated 3D motion based on a comparison between the pseudo real-time 2D image and the real-time 2D image.
